(19) United States
(12) Patent Application Publication
(10) Pub. No.: US 2023/0355536 A1
(43) Pub. Date: Nov. 9, 2023

(54) OZONE-BASED LOW TEMPERATURE SILICON OXIDE COATING FOR PHARMACEUTICAL APPLICATIONS

(71) Applicant: Applied Materials, Inc., Santa Clara, CA (US)

(72) Inventors: Fei Wang, Fremont, CA (US); Geetika Bajaj, Mumbai (IN); Pravin K. Narwankar, Sunnyvale, CA (US)

(21) Appl. No.: 18/199,625

(22) Filed: May 19, 2023

Related U.S. Application Data
(63) Continuation of application No. PCT/US2023/020994, filed on May 4, 2023.
(60) Provisional application No. 63/339,374, filed on May 6, 2022.
(60) Provisional application No. 63/339,377, filed on May 6, 2022.

(51) Int. Cl. A61K 9/50

(52) U.S. Cl. CPC A61K 9/5089; A61K 9/501

(57) ABSTRACT

This disclosure pertains to coated drug compositions and methods of preparing coated drug compositions with a low temperature o-zone based silicon oxide coating. Specifically, the instant application discloses a method to coat active pharmaceutical ingredient particles using a silicon precursor and ozone at a low temperature.

### CROSS-REFERENCE TO RELATED APPLICATIONS

This application is a by-pass continuation application of and claims the benefit of priority to PCT Application No. PCT/US2023-020994, filed on May 4, 2023, which claims priority to U.S. Application No. 63/339,374 and 63/339,377, filed on May 6, 2022 the contents of which are hereby incorporated by reference.

### TECHNICAL FIELD

This disclosure pertains to coated drug compositions and methods of preparing coated drug compositions with a silicon oxide coating at a low temperature.

### BACKGROUND

It is of great interest to the pharmaceutical industry to develop improved formulations of active pharmaceutical ingredients (APIs). Formulation can influence the stability and bioavailability of the APIs as well as other characteristics. Formulation can also influence various aspects of drug product (DP) manufacture, for example, the ease and safety of the manufacturing process.

Recently, we have demonstrated that a metal oxide coating can be applied to API particles using atomic layer coating (ALC). The suitable metal oxide can be aluminum oxide, titanium oxide or zinc oxide.

In the manufacture of drug products, it is also desirable to coat API particles and particles containing API using silicon oxide, a well-accepted inert material, in order to improve the stability and bioavailability of the API. However, in the field of consumer electronics, a silicon oxide coating is usually applied at a high temperature. It is desirable to coat the API particles at a relatively low temperature in order to minimize the damage to the API.

Recently, we have demonstrated that API can be coated with silicon oxide using a silicon precursor (e.g., SiCl4), Tris(tertpentoxy)silanol with a catalyst (e.g., Trimethylaluminium). However, that previous method causes concerns regarding aluminum or chloride contamination. In addition, there is a need for a silicon oxide coating that improves particle characteristics such as dissolution profile and powder flowability.

### SUMMARY

Described herein is a method of preparing a pharmaceutical composition by coating API particles with silicon oxide at a low temperature, the method comprising the sequential steps of: (a) providing particles comprising or consisting of an API; (b) performing atomic layer coating to apply a silicon oxide layer to the particles thereby preparing coated particles comprising or consisting of an API enclosed by silicon oxide; and (c) processing the coated particles to prepare a pharmaceutical composition.

In various embodiments: the step of performing atomic layer coating comprises:
(b1) loading the particles comprising the drug into a reactor;
(b2) applying a vaporous or gaseous silicon precursor to the particles in the reactor;
(b3) performing one or more pump-purge cycles of the reactor using inert gas;
(b4) applying an ozone to the particles in the reactor; and
(b5) performing one or more pump-purge cycles of the reactor using inert gas.

In some embodiments, steps (b2)-(b5) are performed at least four times (e.g., 5, 10, 15, 20, 25 or more times) providing a first, second, third and fourth cycle, etc.

In some embodiments, the silicon oxide coating does not comprise any chloride or HCl.

In one aspect, the disclosure provides a method of preparing a pharmaceutical composition comprising coated particles comprising an active pharmaceutical ingredient (API) enclosed by one or more silicon oxide layers, the method comprising the sequential steps of: (a) Providing uncoated particles comprising an API; (b1) Loading the particles comprising the API into a reactor; (b2) Applying a vaporous or gaseous silicon precursor to the particles in the reactor by pulsing the vaporous or gaseous silicon precursor into the reactor; (b3) Performing one or more pump-purge cycles of the reactor using an inert gas; (b4) Applying an ozone to the particles in the reactor by pulsing the ozone into the reactor; (b5) Performing one or more pump-purge cycles of the reactor using an inert gas; (c) Processing the coated particles to prepare a pharmaceutical composition.

In some embodiments, the uncoated particles are crystalline.

In some embodiments, the silicon oxide coating constitutes 0-10% of the total weight of the coated particles.

In some embodiments, the API is an organic compound.

In some embodiments, steps (b2)-(b5) are performed at least four times providing a first, second, third and fourth cycle.

In some embodiments, a subset of vapor or gaseous content is pumped out prior to step (b3) and/or step (b5).

In some embodiments, the silicon oxide layer on the coated particles has a thickness in the range of 0.1 nm to 120 nm.

In some embodiments, the silicon oxide layer on the coated particles has a thickness in the range of between 10 nm and 50 nm.

In some embodiments, step (c) comprises combining the coated particles with one or more pharmaceutically acceptable excipients.

In some embodiments, steps (b2)-(b5) takes place at a temperature between 25° C. and 100° C.

In some embodiments, steps (b2)-(b5) takes place at a temperature between 35° C. and 50° C.

In some embodiments, step (b4) comprises a holding time in the range of 1 minute to 1 hour.

In some embodiments, step (b2) comprises a holding time in the range of 1 minute to 1 hour.

In some embodiments, the silicon precursor in step (b2) is Diisopropylamino silane (DIPAS).

In some embodiments, the silicon precursor in step (b2) is 1,2-Bis(diisopropylamino)disilane (BDIPADS).

In some embodiments, the ozone in step (b4) is generated using an ozone generator with an oxygen flow rate of 100 sccm.

In some embodiments, step (b1) further comprises agitating the API.

In one aspect, the disclosure provides a pharmaceutical composition prepared by the method described herein.

In some embodiments, the coated particles do not comprise any chloride.

In some embodiments, the coated particles exhibit improved flowability comparing to uncoated particles.

In some embodiments, the coated particles exhibit similar or increased hydrophobicity comparing to uncoated particles.

In some embodiments, the silicon oxide coating is free of HCl and Cl.

In one aspect, the disclosure provides a pharmaceutical composition comprising coated particles comprising an active pharmaceutical ingredient (API) enclosed by one or more silicon oxide layers, wherein the pharmaceutical composition is prepared following the sequential steps of: (a) Providing uncoated particles comprising an API; (b1) Loading the particles comprising the API into a reactor; (b2) Applying a vaporous or gaseous silicon precursor to the particles in the reactor by pulsing the vaporous or gaseous silicon precursor into the reactor; (b3) Performing one or more pump-purge cycles of the reactor using an inert gas; (b4) Applying an ozone to the particles in the reactor by pulsing the ozone into the reactor; (b5) Performing one or more pump-purge cycles of the reactor using an inert gas; (c) Processing the coated particles to prepare a pharmaceutical composition.

In some embodiments, the pharmaceutical composition is free of HCl and Cl.

Unless otherwise defined, all technical and scientific terms used herein have the same meaning as commonly understood by one of ordinary skill in the art to which this invention belongs. Methods and materials are described herein for use in the present invention; other suitable methods and materials known in the art can also be used. The materials, methods, and examples are illustrative only and not intended to be limiting.

Other features and advantages of the invention will be apparent from the following detailed description and figures, and from the claims.

### BRIEF DESCRIPTION OF THE DRAWINGS

FIG. 1A is a schematic illustration of the chemical structure of Diisopropylamino silane (“DIPAS”). FIG. 1B is a schematic illustration of the chemical structure of 1,2-Bis(diisopropylamino)disilane (“BDIPADS”).

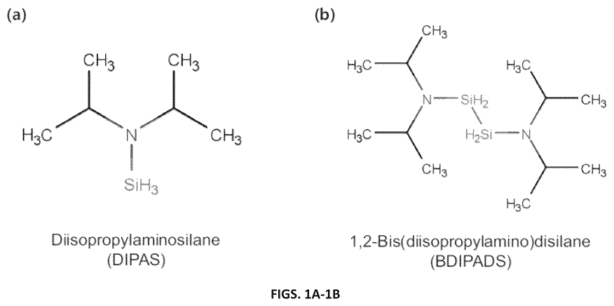

FIGS. 2A-2B (FIG. 2B is a zoom-in image of FIG. 2A) show the FTIR spectrum of the coated and uncoated API particles.

FIGS. 3A-3B (FIG. 3B is a zoom-in image of FIG. 3A) show the TEM cross-section images of the coating on the coated API particles.

FIG. 4 shows the EDS depth profile on the coated API.

FIG. 5 shows the EDS mapping of the coated API.

FIGS. 6A-6B (FIG. 6B is a zoom-in image of FIG. 6A) show the FTIR spectrum of the coated and uncoated API particles.

FIGS. 7A-7B show the TEM cross-section images of the coating on the coated API particles (FIG. 7B is a zoom-in image of FIG. 7A).

FIGS. 8A-8D show the HPLC analysis of the API (acetaminophen) after ozone treatment. FIG. 8A shows the HPLC analysis of uncoated API (acetaminophen). FIG. 8B shows the HPLC analysis of API treated with ozone generated with an ozone generator with an oxygen flow rate of 10 sccm. FIG. 8C shows the HPLC analysis of API treated with ozone generated with an ozone generator with an oxygen flow rate of 30 sccm. FIG. 8D shows the HPLC analysis of API treated with ozone generated with an ozone generator with an oxygen flow rate of 50 sccm.

FIGS. 9A-9B (FIG. 9B is a zoom-in image of FIG. 9A) show the FTIR spectrum of the coated and uncoated API particles.

FIG. 10 shows the FTIR spectrum of the coated API particles.

FIGS. 11A-11C show the image of dd water drops on the coated particles. FIG. 11A shows the image of a dd water drop on the coated particles with ozone-based coating (BD-9). FIG. 11B shows the image of a dd water drop on the coated particles with water-based coating (BD-9-BD-water). FIG. 11C shows the image of a dd water drop on the coated particles with TMA-ozone based process (BD-9-TMA-O3).

FIG. 12 is a schematic illustration of an exemplary reactor system.

### DETAILED DESCRIPTION

The present disclosure provides methods of preparing pharmaceutical compositions comprising particles comprising an API coated with silicon oxide. The coating is of controlled thickness. Because the coating is relatively thin, drug products with high drug loading can be produced. For example, the silicon oxide layer can have a thickness in range of 0.1 nm to 100 nm. Finally, there are benefits with respect to cost and ease of manufacture because multiple coatings can be applied in the same reactor.

### Drug

The term “drug,” in its broadest sense includes all small molecule (e.g., non-biologic) APIs, in particular APIs that are organic molecules. The drug could be selected from the group consisting of an analgesic, an anesthetic, an anti-inflammatory agent, an anthelmintic, an anti-arrhythmic agent, an antiasthma agent, an antibiotic, an anticancer agent, an anticoagulant, an antidepressant, an antidiabetic agent, an antiepileptic, an antihistamine, an antitussive, an antihypertensive agent, an antimuscarinic agent, an antimycobacterial agent, an antineoplastic agent, an antioxidant agent, an antipyretic, an immunosuppressant, an immunostimulant, an antithyroid agent, an antiviral agent, an anxiolytic sedative, a hypnotic, a neuroleptic, an astringent, a bacteriostatic agent, a beta-adrenoceptor blocking agent, a blood product, a blood substitute, a bronchodilator, a buffering agent, a cardiac inotropic agent, a chemotherapeutic, a contrast media, a corticosteroid, a cough suppressant, an expectorant, a mucolytic, a diuretic, a dopaminergic, an antiparkinsonian agent, a free radical scavenging agent, a growth factor, a haemostatic, an immunological agent, a lipid regulating agent, a muscle relaxant, a parasympathomimetic, a parathyroid calcitonin, a biphosphonate, a prostaglandin, a radio-pharmaceutical, a hormone, a sex hormone, an anti-allergic agent, an appetite stimulant, an anoretic, a steroid, a sympathomimetic, a thyroid agent, a vaccine, a vasodilator and a xanthine.

Exemplary types of small molecule drugs include, but are not limited to, acetaminophen, clarithromycin, azithromycin, ibuprofen, fluticasone propionate, salmeterol, pazopanib HCl, palbociclib, and amoxicillin potassium clavulanate.

### Atomic Layer Coating (ALC)

In the atomic layer coating method (also referred to as atomic layer deposition (ALD)), a thin film coating is formed on the surface of a particle by depositing successive atomic layers of one or more coating materials. In some embodiment, the coating material is silicon oxide.

### Reactor System

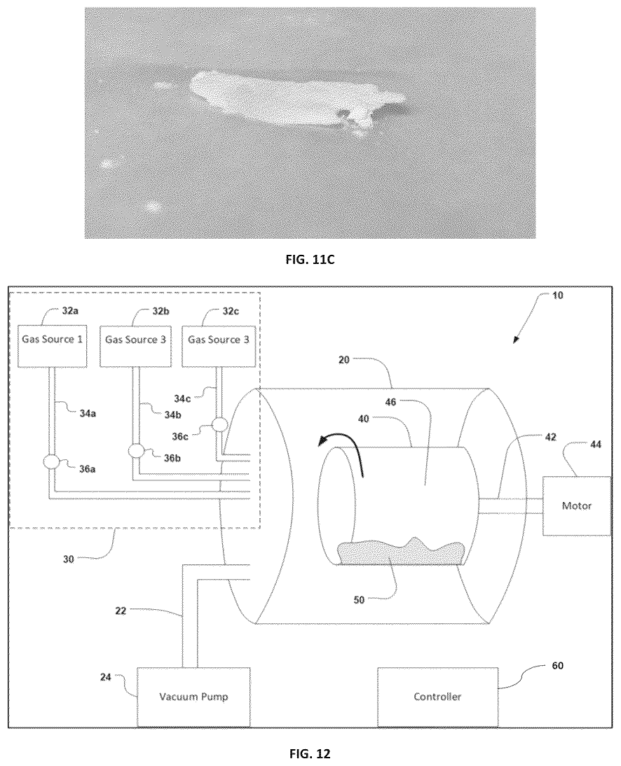

The term “reactor system” in its broadest sense includes all systems that could be used to perform ALC. An exemplary reactor system is illustrated in FIG. 12 and further described below.

FIG. 12 illustrates a reactor system 10 for performing coating of particles, with thin-film coatings. The reactor system 10 can perform ALC coating. The reactor system 10 permits ALC coating to be performed at higher (above 50° C., e.g., 50-100° C. or higher) or lower processing temperature, e.g., below 50° C., e.g., at or below 35° C. For example, the reactor system 10 can form thin-film silicon oxide on the particles primarily by ALC at temperatures of 40-80° C., e.g., 40° C. or 80° C. In general, the particles can remain or be maintained at such temperatures. This can be achieved by having the reactants and/or the interior surfaces of the reactor chamber (e.g., the chamber 20 and drum 40 discussed below) remain or be maintained at such temperatures.

Again, illustrating an ALC process, the reactor system 10 includes a stationary vacuum chamber 20 which is coupled to a vacuum pump 24 by vacuum tubing 22. The vacuum pump 24 can be an industrial vacuum pump sufficient to establish pressures less than 1 Torr, e.g., 1 to 100 mTorr, e.g., 50 mTorr. The vacuum pump 24 permits the chamber 20 to be maintained at a desired pressure and permits removal of reaction byproducts and unreacted process gases.

In operation, the reactor 10 performs the ALC thin-film coating process by introducing a gaseous oxidant and silicon precursor into the chamber 20. The gaseous oxidant and silicon precursor are spiked alternatively into the reactor. In addition, the ALC reaction can be performed at low temperature conditions, such as below 80° C., e.g., below 50° C.

The chamber 20 is also coupled to a chemical delivery system 30. The chemical delivery system 30 includes three or more gas sources 32a, 32b, 32c coupled by respective delivery tubes 34a, 34b, 34c and controllable valves 36a, 36b, 36c to the vacuum chamber 20. The chemical delivery system 30 can include a combination of restrictors, gas flow controllers, pressure transducers, and ultrasonic flow meters to provide controllable flow rate of the various gasses into the chamber 20. The chemical delivery system 30 can also include one or more temperature control components, e.g., a heat exchanger, resistive heater, heat lamp, etc., to heat or cool the various gasses before they flow into the chamber 20. Although FIG. 12 illustrates separate gas lines extending in parallel to the chamber for each gas source, two or more of the gas lines could be joined, e.g., by one or more three-way valves, before the combined line reaches the chamber 20.

One of the gas sources can provide an oxidant. In particular, a gas source can provide a vaporous or gaseous oxidant. For example, the oxidant can be ozone generated by an ozone generator. As another example, the oxidant can be water vapor.

One of the gas sources can be a silicon precursor. In particular, a gas source can provide a vaporous or gaseous silicon precursor. For example, the silicon precursor can be Diisopropylamino silane (DIPAS) or 1,2-Bis(diisopropylamino)disilane (BDIPADS).

One of the gas sources can provide a purge gas. In particular, the third gas source can provide a gas that is chemically inert to the oxidant and silicon precursor, the coating, and the particles being processed. For example, the purge gas can be N₂, or a noble gas, such as argon.

A rotatable coating drum 40 is held inside the chamber 20. The drum 40 can be connected by a drive shaft 42 that extends through a sealed port in a side wall of the chamber to a motor 44. The motor 44 can rotate the drum at speeds of 1 to 100 rpm. Alternatively, the drum can be directly connected to a vacuum source through a rotary union.

The particles to be coated, shown as a particle bed 50, are placed in an interior volume 46 of the drum 40. The drum 40 and chamber 20 can include sealable ports (not illustrated) to permit the particles to be placed into and removed from the drum 40.

The body of the drum 40 is provided by one or more of a porous material, a solid metal, and a perforated metal. The pores through the cylindrical side walls of the drum 40 can have a dimension of 10 μm.

In operation, one of the gasses flows into chamber 20 from the chemical delivery system 30 as the drum 40 rotates. A combination of pores (1-100 um), holes (0.1-10 mm), or large openings in the coating drum 40 serve to confine the particles in the coating drum 40 while allowing rapid delivery of precursor chemistry and the pumping of byproducts or unreacted species. Due to the pores in the drum 40, the gas can flow between the exterior of the drum 40, i.e., the reactor chamber 20, and the interior of the drum 40. In addition, rotation of the drum 40 agitates the particles to keep them separate, ensuring a large surface area of the particles remains exposed. This permits fast, uniform interaction of the particle surface with the process gas.

In some implementations, one or more temperature control components are integrated into the drum 40 to permit control of the temperature of the drum 40. For example, a resistive heater, a thermoelectric cooler, or other component can be in or on the side walls of the drum 40.

The reactor system 10 also includes a controller 60 coupled to the various controllable components, e.g., vacuum pump 24, gas distribution system 30, motor 44, a temperature control system, etc., to control operation of the reactor system 10. The controller 60 can also be coupled to various sensors, e.g., pressure sensors, flow meters, etc., to provide closed loop control of the pressure of the gasses in the chamber 20.

In general, the controller 60 can operate the reactor system 10 in accord with a “recipe.” The recipe specifies an operating value for each controllable element as a function of time. For example, the recipe can specify the times during which the vacuum pump 24 is to operate, the times of and flow rate for each gas source 32a, 32b, 32c, the rotation rate of the motor 44, etc. The controller 60 can receive the recipe as computer-readable data (e.g., that is stored on a non-transitory computer readable medium).

The controller 60 and other computing device parts of systems described herein can be implemented in digital electronic circuitry, or in computer software, firmware, or hardware. For example, the controller can include a processor to execute a computer program as stored in a computer program product, e.g., in a non-transitory machine-readable storage medium. Such a computer program (also known as a program, software, software application, or code) can be written in any form of programming language, including compiled or interpreted languages, and it can be deployed in any form, including as a standalone program or as a module, component, subroutine, or other unit suitable for use in a computing environment. In some implementations, the controller 60 is a general-purpose programmable computer. In some implementations, the controller can be implemented using special purpose logic circuitry, e.g., an FPGA (field programmable gate array) or an ASIC (application specific integrated circuit).

### Operation

Initially, particles are loaded into the drum 40 in the reactor system 10. The particles can be purely particles of a drug (or a combination of particles of a first drug and a second drug) or a mixture of particles of a drug (or a combination of particles of a first drug and a second drug) and particles of an excipient. In some cases, the particles are composed of one or more drugs (e.g., one of the drugs discussed above) and one or more excipients. Once any access ports are sealed, the controller 60 operates the reactor system 10 according to the recipe in order to form the thin-film silicon oxide on the particles.

In particular, the oxidant and the silicon precursor can be alternately supplied to the chamber 20, with each step of supplying an oxidant or the silicon precursor followed by a purge cycle in which the inert gas is supplied to the chamber 20 to force out the excessive oxidant or silicon precursor and by-products used in the prior step. Moreover, one or more of the gases (silicon precursor gases and/or the inert gas and/or oxidant gas) can be supplied in pulses in which the chamber 20 is filled with the gas to a specified pressure, a holding time is permitted to pass, and the chamber is evacuated by the vacuum pump 24 before the next pulse commences.

In particular, the controller 60 can operate the reactor system 10 as follows.

In a silicon precursor half-cycle, while the motor 44 rotates the drum 40 to agitate the particles 50:
i) The gas distribution system 30 is operated to flow the silicon precursor gas, e.g., DIPAS, from the source 32a into the chamber 20 until a first specified pressure is achieved. The specified pressure can be 0.1 Torr to half of the saturation pressure of the silicon precursor gas.
ii) Flow of the silicon precursor is halted, and a specified holding time is permitted to pass, e.g., as measured by a timer in the controller. This permits the silicon precursor to flow through the particle bed in the drum 40 and react with the surface of the particles 50 inside the drum 40.
iii) The vacuum pump 50 evacuates the chamber 20, e.g., down to pressures below 1 Torr, e.g., to 1 to 100 mTorr, e.g., 50 mTorr.

Next, in a first purge cycle, while the motor 44 rotates the drum to agitate the particles 50:
iv) The gas distribution system 30 is operated to flow the inert gas, e.g., N₂, from the source 32c into the chamber 20 until a second specified pressure is achieved. The second specified pressure can be 1 to 100 Torr.
v) Flow of the inert gas is halted, and a specified delay time is permitted to pass, e.g., as measured by the timer in the controller. This permits the inert gas to flow through the pores in the drum 40 and diffuse through the particles 50 to displace the silicon precursor gas and any vaporous by-products.
vi) The vacuum pump 50 evacuates the chamber 20, e.g., down to pressures below 1 Torr, e.g., to 1 to 500 mTorr, e.g., 50 mTorr.

These steps (iv)-(vi) can be repeated a number of times set by the recipe, e.g., six to twenty times, e.g., sixteen times.

In a oxidant half-cycle, while the motor 44 rotates the drum 40 to agitate the particles 50:
vii) The gas distribution system 30 is operated to flow the oxidant, e.g., ozone generated from an ozone generator, from the source 32a into the chamber 20 until a third specified pressure is achieved. The third pressure can be 0.1 Torr to half of the saturation pressure of the oxidant gas.
viii) Flow of the oxidant is halted, and a specified holding time is permitted to pass, e.g., as measured by the timer in the controller. This permits the oxidant to flow through the pores in the drum 40 and react with the surface of the particles 50 inside the drum 40.
ix) The vacuum pump 50 evacuates the chamber 20, e.g., down to pressures below 1 Torr, e.g., to 1 to 500 mTorr, e.g., 50 mTorr.

Next, a second purge cycle is performed. This second purge cycle can be identical to the first purge cycle, or can have a different number of repetitions of the steps (iv)-(vi) and/or different delay time and/or different pressure.

The cycle of the silicon precursor half-cycle, first purge cycle, oxidant half cycle and second purge cycle can be repeated a number of times set by the recipe, e.g., one to ten times.

As noted above, the coating process can be performed at low processing temperature, e.g., below 80° C., e.g., at or below 50° C. In particular, the particles can remain or be maintained at such temperatures during all of steps (i)-(ix) noted above. In general, the temperature of the interior of the reactor chamber does not exceed 80° C. during of steps (i)-(ix). This can be achieved by having the oxidant gas, silicon precursor gas and inert gas be injected into the chamber at such temperatures during the respective cycles. In addition, physical components of the chamber can remain or be maintained at such temperatures, e.g., using a cooling system, e.g., a thermoelectric cooler, if necessary.

### Process for Preparing Pharmaceutical Compositions Comprising Drugs Encapsulated by Silicon Oxide

Provided are two exemplary methods for a pharmaceutical composition comprising a drug-containing core enclosed by silicon oxide. The first exemplary method includes the sequential steps of: (a) loading the particles comprising the drug into a reactor, (b) applying a vaporous or gaseous silicon precursor to the substrate in the reactor, (c) performing one or more pump-purge cycles of the reactor using inert gas, (d) applying a vaporous or gaseous oxidant (e.g., ozone) to the substrate in the reactor, and (e) performing one or more pump-purge cycles of the reactor using inert gas. In some embodiments of the first exemplary method, the sequential steps (b)-(e) are optionally repeated one or more times to increase the total thickness of the silicon oxide that enclose the solid core of the coated particles. In some embodiments, the reactor pressure is allowed to stabilize following step (a), step (b), and/or step (d). In some embodiments, the reactor contents are agitated prior to and/or during step (b), step (c), and/or step (e). In some embodiments, a subset of vapor or gaseous content is pumped out prior to step (c) and/or step (e).

The second exemplary method includes (e.g., consists of) the sequential steps of (a) loading the particles comprising the drug into a reactor, (b) reducing the reactor pressure to less than 50 m Torr, (c) agitating the reactor contents until the reactor contents have a desired moisture content, (d) pressurizing the reactor to at least 0.3 Torr by adding a vaporous or gaseous silicon precursor, (e) allowing the reactor pressure to stabilize, (f) agitating the reactor contents, (g) pumping out a subset of vapor or gaseous content and determining when to stop pumping based on analysis of content in reactor, (h) performing a sequence of pump-purge cycles of the reactor using insert gas, (i) pressuring the reactor to 8 Torr by adding a vaporous or gaseous oxidant (e.g., ozone), (j) allowing the reactor pressure to stabilize, (k) agitating the reactor contents, (l) pumping out a subset of vapor or gaseous content and determining when to stop pumping based on analysis of content in reactor and (m) performing a sequence of pump-purge cycles of the reactor using insert gas. In some embodiments of the second exemplary method, the sequential steps (b)-(m) are optionally repeated one or more times to increase the total thickness of the one or more silicon oxide materials that enclose the solid core of the coated particles.

Some embodiments provide a method of preparing a pharmaceutical composition comprising coated particles comprising an active pharmaceutical ingredient enclosed by silicon oxide, the method comprising the sequential steps of: (a) providing uncoated particles comprising an active pharmaceutical ingredient (API); (b) performing atomic layer coating to apply a silicon oxide layer to uncoated particles comprising an active pharmaceutical ingredient thereby preparing coated particles comprising an active pharmaceutical ingredient enclosed by silicon oxide; (c) processing the coated particles to prepare a pharmaceutical composition wherein the processing comprising combining the particles with one or more pharmaceutically acceptable (e.g., acceptable in an oral drug product) excipients; and (d) processing the pharmaceutical composition to form a drug product (e.g, a pill, tablet or capsule). In some cases, the drug product is an oral drug product.

In some embodiments, the uncoated particles are at least 50% wt/wt API. In some embodiments, the uncoated particles are at least 70%, 80%, 90%, 99% or 100% wt/wt API. In some cases, the API is crystalline. In some embodiments, the coated particles have a D50 of 0.1 μm to 200 μm or 0.1 μm to 1 μm or 0.1 μm to 10 μm 0.1 μm to 50 μm on a volume average basis. In some embodiments, the coated particles have a D90 of 200 μm to 2000 μm on a volume average basis. In some embodiments, the uncoated particles have a D50 of 0.1 μm to 200 μm or 0.1 μm to 1 μm or 0.1 μm to 10 μm 0.1 μm to 50 μm on a volume average basis. In some embodiments, the uncoated particles have a D90 of 200 μm to 2000 μm on a volume average basis.

In some embodiments, the silicone oxide coating is a solid (pinhole-free) conformal coating.

In some embodiments, the step of performing atomic layer coating comprises: (b1) loading the particles comprising the drug into a reactor; (b2) applying a vaporous or gaseous silicon precursor to the particles in the reactor; (b3) performing one or more pump-purge cycles of the reactor using inert gas; (b4) applying a vaporous or gaseous oxidant (e.g., ozone) to the particles in the reactor; and (b5) performing one or more pump-purge cycles of the reactor using inert gas. In some embodiments, steps (b2)-(b5) are performed two or more times to increase the total thickness of the silicon oxide layer before step (c) is performed.

In some embodiments, the reactor pressure is allowed to stabilize following step (b1), step (b2), and/or step (b4). In some embodiments, the reactor contents are agitated prior to and/or during step (b1), step (b3), and/or step (b5). In some embodiments, a subset of vapor or gaseous content is pumped out prior to step (b3) and/or step (b5). In some embodiments, step (b) takes place at a temperature between 35° C. and 50° C. In some embodiments, step (c) comprises combining the coated particles with one or more pharmaceutically acceptable excipients.

In some embodiments, the silicon oxide layer has a thickness in range of 0.1 nm to 100 nm or 0.1 nm to 10 nm or 0.1 nm to 50 nm.

Some embodiments provide a pharmaceutical composition comprising coated particles comprising an active pharmaceutical ingredient enclosed by silicon oxide, prepared by a method comprising the sequential steps of: (a) providing uncoated particles comprising an active pharmaceutical ingredient; (b) performing atomic layer coating to apply a silicon oxide layer to uncoated particles comprising an active pharmaceutical thereby preparing coated particles comprising an active pharmaceutical ingredient enclosed by silicon oxide; and (c) processing the coated particles to prepare a pharmaceutical composition.

In some embodiments, the step of performing atomic layer coating comprises:
(b1) loading the particles comprising the drug into a reactor; (b2) applying a vaporous or gaseous silicon precursor to the particles in the reactor; (b3) performing one or more pump-purge cycles of the reactor using inert gas; (b4) applying a vaporous or gaseous oxidant (e.g., ozone) to the particles in the reactor; and (b5) performing one or more pump-purge cycles of the reactor using inert gas.

In some embodiments, steps (b2)-(b5) are performed two or more times to increase the total thickness of the silicon oxide layer before step (c) is performed. In some embodiments, the particles are agitated prior to and/or during step (a). In some embodiments, the reactor pressure is allowed to stabilize following step (b1), step (b2), and/or step (b4). In some embodiments, the reactor contents are agitated prior to and/or during step (b1), step (b3), and/or step (b5). In some embodiments, a subset of vapor or gaseous content is pumped out prior to step (b3) and/or step (b5). In some embodiments, step (b) takes place at a temperature between 35° C. and 50° C.

In some embodiments, the silicon oxide layer has a thickness in range of 0.1 nm to 100 nm. In some embodiments, the uncoated particles have a median particle size, on a volume average basis between 0.1 μm and 1000 μm.

In some embodiments, the coated particles comprising an active pharmaceutical ingredient further comprise one or more pharmaceutically acceptable excipients. In some embodiments, the uncoated particles consist of the active pharmaceutical ingredient.

In some embodiments, the coated particles exhibit increased hydrophobicity comparing to the uncoated particles.

In some embodiments, the coated particles generated by the instant ozone-based method exhibit increased hydrophobicity comparing to 1) SiO2 atomic layer coating using a catalyst (e.g., Trimethylaluminium) and a silicon precursor (e.g., Tris(tertpentoxy)silanol) or 2) SiO2 atomic layer coating using SiCl4 with water.

In some embodiments, the coated particles exhibit increased powder flowability (“FF”) comparing to the uncoated particles.

In some embodiments, the coated particles generated by the instant ozone-based method exhibit increased powder flowability comparing to 1) SiO2 atomic layer coating using a catalyst (e.g., Trimethylaluminium) and a silicon precursor (Tris(tertpentoxy)silanol) or 2) SiO2 atomic layer coating using SiCl4 with water.

### Pharmaceutically Acceptable Excipients, Diluents, and Carriers

Pharmaceutically acceptable excipients include, but are not limited to:
(1) surfactants and polymers including: polyethylene glycol (PEG), polyvinylpyrrolidone (PVP), sodium lauryl sulfate, polyvinylalcohol, crospovidone, polyvinylpyrrolidone-polyvinylacrylate copolymer (PVPVA), cellulose derivatives, hydroxypropylmethyl cellulose, hydroxypropyl cellulose, carboxymethylethyl cellulose, hydroxypropyllmethyl cellulose phthalate, polyacrylates and polymethacrylates, urea, sugars, polyols, carbomer and their polymers, emulsifiers, sugar gum, starch, organic acids and their salts,
(2) binding agents such as cellulose, cross-linked polyvinylpyrrolidone, microcrystalline cellulose;
(3) filling agents such as lactose monohydrate, lactose anhydrous, microcrystalline cellulose and various starches;
(4) lubricating agents such as agents that act on the flowability of a powder to be compressed, including colloidal silicon dioxide, talc, stearic acid, magnesium stearate, calcium stearate, silica gel;
(5) sweeteners such as any natural or artificial sweetener including sucrose, xylitol, sodium saccharin, cyclamate, aspartame, and acesulfame K;
(6) flavoring agents;
(7) preservatives such as potassium sorbate, methylparaben, propylparaben, benzoic acid and its salts, other esters of parahydroxybenzoic acid such as butylparaben, alcohols such as ethyl or benzyl alcohol, phenolic chemicals such as phenol, or quarternary compounds such as benzalkonium chloride;
(8) buffers;
(9) diluents such as pharmaceutically acceptable inert fillers, such as microcrystalline cellulose, lactose, dibasic calcium phosphate, saccharides, and/or mixtures of any of the foregoing;
(10) wetting agents such as corn starch, potato starch, maize starch, and modified starches, and mixtures thereof;
(11) disintegrants; such as croscarmellose sodium, crospovidone, sodium starch glycolate; and
(12) effervescent agents such as effervescent couples such as an organic acid (e.g., citric, tartaric, malic, fumaric, adipic, succinic, and alginic acids and anhydrides and acid salts), or a carbonate (e.g., sodium carbonate, potassium carbonate, magnesium carbonate, sodium glycine carbonate, L-lysine carbonate, and arginine carbonate) or bicarbonate (e.g. sodium bicarbonate or potassium bicarbonate).

### EXAMPLES

The following materials and methods were used in the Examples.

### Method for ALC Coating

In brief, in one embodiment, the method for creating a silicon oxide coating comprised the sequential steps of:
(a) Loading particles comprising the drug (API) into a rotatory reactor;
(b) Pulsing a silicon precursor with a holding time of 5 minutes;
(c) Purging the reactor with an inert gas to remove the silicon precursor;
(d) Pulsing ozone into the reactor, with a holding time of 10 minutes;
(e) Purging the reactor with an inert gas to remove extra ozone.

In some cases, the steps of (b)-(e) were repeated more than once to increase the total thickness of the silicon oxide that enclose the drug particle core.

### Example 1: Silicon Oxide Coating at 50° C.

API (Acetaminophen) particles were coated with silicon oxide at 50° C. for 40 cycles following the methods described in Table 1 below:

TABLE 1

| BDIPADS | O₃ (O₂ flowrate | BDIPADS hold | O₃ hold |
|---|---|---|---|
| (pressure) | and pressure) | time | time |
| 0.3 torr | 100 sccm, 17 torr | 5 min | 10 min |

In step (a), 3 gram of API was loaded to the rotatory reactor (rotating at 10-100 rpm). The rotatory reactor is beneficial because it can better expose the API particles. In step (b), the silicon precursor (BDIPADS) was pulsed into the reactor at about 0.3 torr, with a holding time of 5 minutes and a reaction temperature of 50° C. After the 5-minute holding time, in step (c), the reactor was purged using an inert gas to remove excessive silicon precursors. In step (d), ozone (generated by an ozone generator with an oxygen flow rate of 100 sccm and 17 torr) was pulsed into the reactor with a holding time of 10 minutes and a reaction temperature of 50° C. After the 10-minute holding time, in step (e), the reactor was purged using an inert gas to remove excessive ozone. Steps (b)-(e) were repeated 40 times.

### Fourier Transform Infrared Spectroscopy (FTIR) Analysis

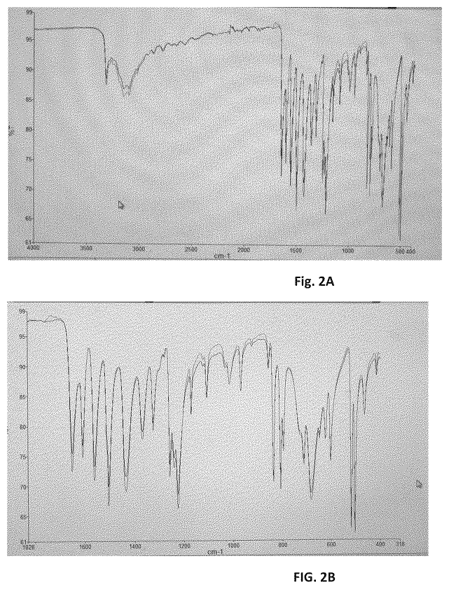

FIGS. 2A-2B (FIG. 2B is a zoom-in image of FIG. 2A) show the FTIR spectrum of the coated and uncoated acetaminophen API particles. As shown in FIGS. 2A-2B, there are no significant change in FTIR signals before and after the ozone-based coating process. This result indicates that there are no change in the chemical bonding information, and that the API was not damaged by the ozone-based coating process.

### Transmission Electron Microscopy (TEM) Analysis

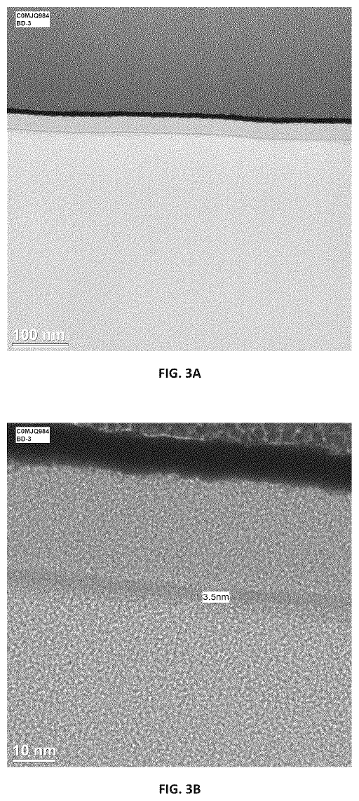

FIGS. 3A-3B (FIG. 3B is a zoom-in image of FIG. 3A) show the TEM cross-section images of the coating on the coated API particles. The TEM image shows a coating layer of about 3.5 nm on the coated API particles.

### Energy-Dispersive X-Ray Spectroscopy (EDS) Analysis

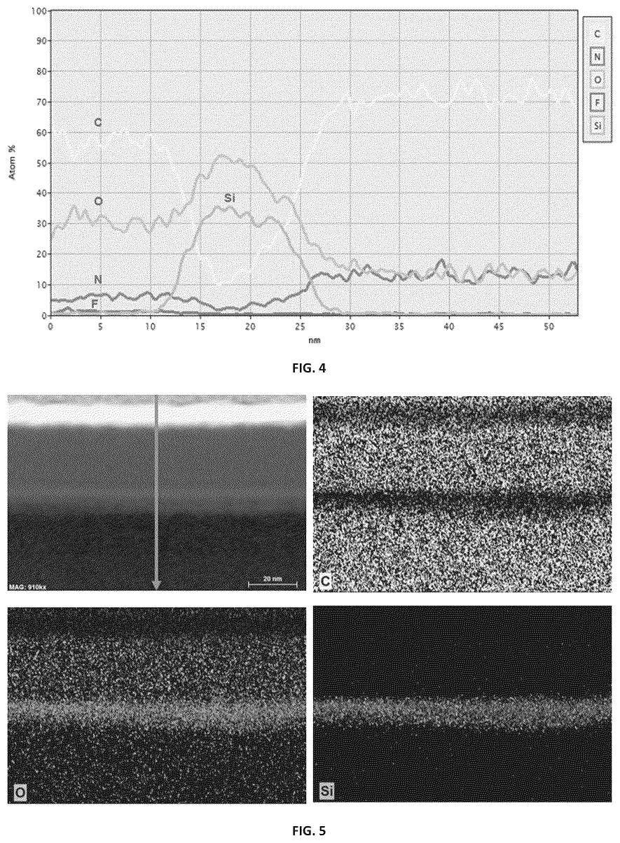

FIG. 4 shows the EDS depth profile on TEM cutting surface of the coated API. The EDS depth profile shows the presence of a silicon oxide layer (Si and O).

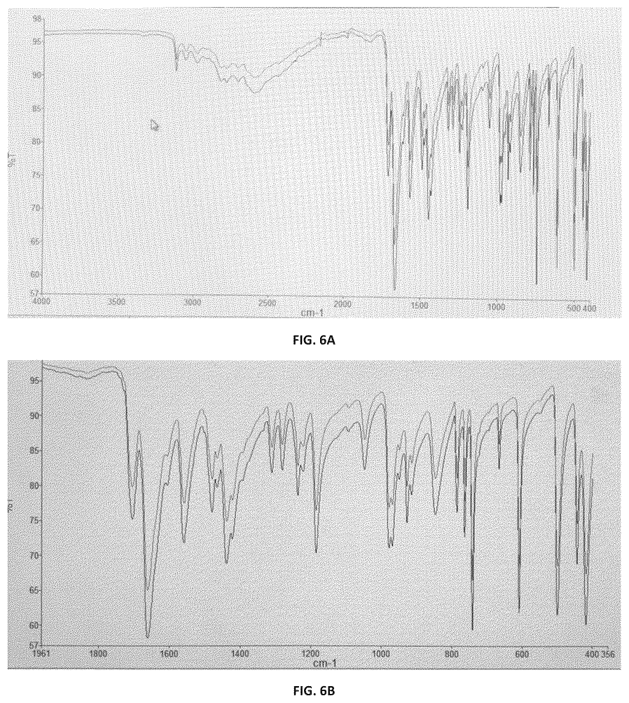

FIG. 5 shows the EDS mapping of TEM cutting surface of the coated API. The EDS mapping also shows the presence of a silicon oxide layer (Si and O).

### X-Ray Photoelectron Spectroscopy (XPS) Analysis

Table 2 shows the XPS data on the coated particles.

TABLE 2

| Sample ID | C | N | O | F | Al | Si | Cl |
|---|---|---|---|---|---|---|---|
| BD-3 | 22.7 | 3.4 | 54.6 | — | — | 19.3 | — |

Table 2 shows atomic concentrations in atomic %. The data in Table 2 are normalized to 100% of the elements detected. A dash line “-” indicates the element is not detected. The data in Table 2 suggests that the coating on the silicon wafer is SiO2. The C, N, O are from API.

### Example 2: Silicon Oxide Coating at 40° C.

API (theophylline) was coated with silicon oxide at 40° C. for 20 cycles following the methods described in Table 1.

In step (a), 1 gram of API was loaded to the rotatory reactor (rotating at 10-100 rpm). In step (b), the silicon precursor (BDIPADS) was pulsed into the reactor at about 0.3 torr, with a holding time of 5 minutes and a reaction temperature of 40° C. After the 5-minute holding time, in step (c), the reactor was purged using an inert gas to remove excessive silicon precursors. In step (d), ozone (generated by an ozone generator with an oxygen flow rate of 100 sccm and 17 torr) was pulsed into the reactor with a holding time of 10 minutes and a reaction temperature of 40° C. After the 10-minute holding time, in step (e), the reactor was purged using an inert gas to remove excessive ozone. Steps (b)-(e) were repeated 20 times.

### Fourier Transform Infrared Spectroscopy (FTIR) Analysis

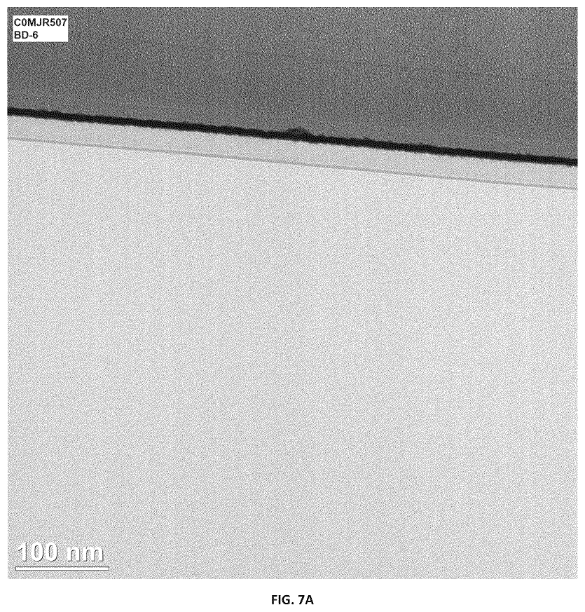

FIGS. 6A-6B (FIG. 6B is a zoom-in image of FIG. 6A) show the FTIR spectrum of the coated and uncoated theophylline API particles. As shown in FIGS. 6A-6B, there are no significant change in FTIR signals before and after the ozone-based coating process. This result indicates that there are no change in the chemical bonding information, and that the API was not damaged by the ozone-based coating process.

### Transmission Electron Microscopy (TEM) Analysis

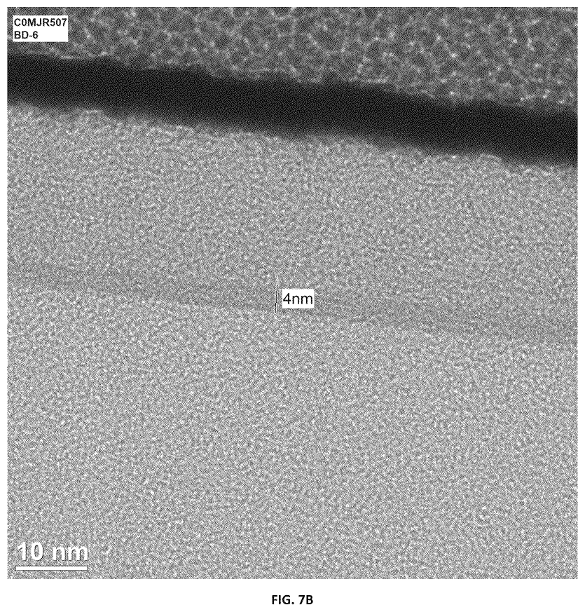

FIGS. 7A-7B show the TEM cross-section images of the coating on the coated theophylline API particles (FIG. 7B is a zoom-in image of FIG. 7A). The TEM image shows a coating layer of about 4 nm on the coated API particles.

### Example 3: Silicon Oxide Coating at 35° C.

API-1 was coated with silicon oxide at 35° C. following the methods described in Table 3.

TABLE 3

| | TMA/water | SiO2 process | FF | TGA % | BD | TD |
|---|---|---|---|---|---|---|
| API-1 | / | / | 2.1 | / | 0.099 | 0.179 |
| BD-11 | 5 cycles | 60 cycles | 7.63 | 3.71 | 0.132 | 0.239 |
| BD-13 | 0 cycles | 65 cycles | 6.22 | 2.83 | 0.15 | 0.192 |

Regarding BD-13, in step (a), 3 gram of API was loaded to the rotatory reactor (rotating at 10-100 rpm). In step (b), the silicon precursor (BDIPADS) was pulsed into the reactor at about 0.3 torr, with a holding time of 5 minutes and a reaction temperature of 35° C. After the 5-minute holding time, in step (c), the reactor was purged using an inert gas to remove excessive silicon precursors. In step (d), ozone (generated by an ozone generator with an oxygen flow rate of 100 sccm and 17 torr) was pulsed into the reactor with a holding time of 10 minutes and a reaction temperature of 35° C. After the 10-minute holding time, in step (e), the reactor was purged using an inert gas to remove excessive ozone. Steps (b)-(e) were repeated 65 times.

Regarding BD-11, in order to protect the API from ozone exposure, the API was coated using a Trimethylaluminium (TMA) based process for five cycles before the ozone-based coating process. To maintain the same number of cycles (65 cycles), BD-11 was then coated using the ozone-based coating process for 60 cycles.

For the TMA-based coating of BD-11, in step (a), 1.5 gram of API-1 was loaded to the rotatory reactor (rotating at 10-100 rpm). In step (b), the catalyst TMA was pulsed into the reactor at about 1 torr, with a holding time of 30 seconds and a reaction temperature of 35° C. After the 30-second holding time, in step (c), the reactor was purged using an inert gas to remove excessive TMA. In step (d), water was pulsed into the reactor at about 1 torr, hold for second with a reaction temperature of 35° C., in step (e), the reactor was purged using an inert gas to remove excessive water Steps (b)-(e) were repeated 5 times.

For the ozone-based coating of BD-11, in step (a), the API with five cycles of TMA-based coating was loaded to the rotatory reactor (rotating at 10-100 rpm). In step (b), the silicon precursor (BDIPADS) was pulsed into the reactor at about 0.3 torr, with a holding time of 5 minutes and a reaction temperature of 35° C. After the 5-minute holding time, in step (c), the reactor was purged using an inert gas to remove excessive silicon precursors. In step (d), ozone (generated by an ozone generator with an oxygen flow rate of 100 sccm and 17 torr) was pulsed into the reactor with a holding time of 10 minutes and a reaction temperature of 35° C. After the 10-minute holding time, in step (e), the reactor was purged using an inert gas to remove excessive ozone. Steps (b)-(e) were repeated 60 times.

After the ozone-based coating process, the uncoated and coated API-1 particles were tested for flowability (“FF”), Thermogravimetric Analysis (“TGA %”), bulk density (“BD”) and tap density (“TD”). The testing results are shown in Table 3.

### Fourier Transform Infrared Spectroscopy (FTIR) Analysis

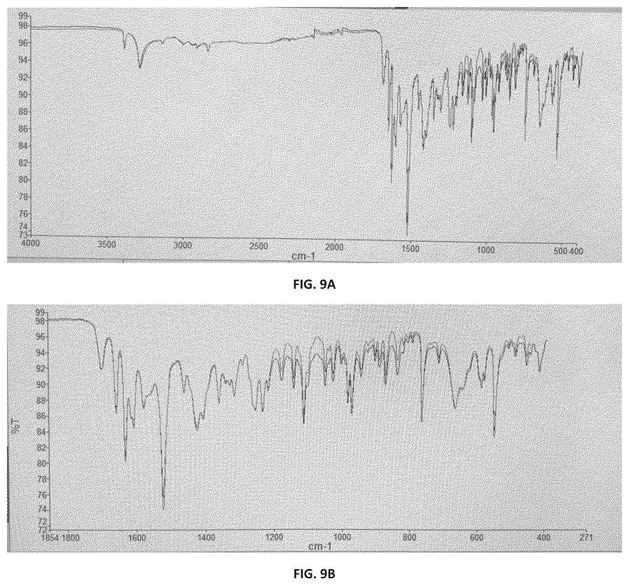

FIGS. 9A-9B (FIG. 9B is a zoom-in image of FIG. 9A) show the FTIR spectrum of the coated and uncoated API-1 particles. As shown in FIGS. 9A-9B, there are no significant change in FTIR signals before and after the ozone-based coating process. This result indicates that there are no change in the chemical bonding information, and that the API was not damaged by the ozone-based coating process.

### Example 4: Silicon Oxide Coating at 35° C.

API-2 was coated with silicon oxide at 35° C. following the methods described in Table 4.

TABLE 4

| | TMA/ | | cohesive | UYS, | | | |
|---|---|---|---|---|---|---|---|
| | water | BD + O₃ | Kpa | KPA | MPS Kp | FF | TGA % |
| API-2 | / | / | 0.998 | 3.1 | 6.04 | 1.62 | / |
| BD-12 | 5 | 60 | 0.375 | 1.21 | 4.85 | 4.02 | 2.95% |

Regarding BD-12, in order to protect the API from ozone exposure, the API was coated using a Trimethylaluminium (TMA) based process for five cycles before the ozone-based coating process.

Regarding the TMA-based process for BD-12, in step (a), 2 gram of API-2 was loaded to the rotatory reactor (rotating at 10-100 rpm). In step (b), the catalyst TMA was pulsed into the reactor at about 1 torr, with a holding time of 30 seconds and a reaction temperature of 35° C. After the 30-second holding time, in step (c), the reactor was purged using an inert gas to remove excessive TMA. In step (d), water was pulsed into the reactor at about 1 torr, 30 second hold time, with a reaction temperature of 35° C. in step (e), the reactor was purged using an inert gas to remove excessive TPS. Steps (b)-(e) were repeated 5 times.

For the ozone-based coating of BD-12, in step (a), the API-2 particles with five cycles of TMA-based coating was loaded to the rotatory reactor (rotating at 10-100 rpm). In step (b), the silicon precursor (BDIPADS) was pulsed into the reactor at about 0.3 torr, with a holding time of 5 minutes and a reaction temperature of 35° C. After the 5-minute holding time, in step (c), the reactor was purged using an inert gas to remove excessive silicon precursors. In step (d), ozone (generated by an ozone generator with an oxygen flow rate of 100 sccm and 17 torr) was pulsed into the reactor with a holding time of 10 minutes and a reaction temperature of 35° C. After the 10-minute holding time, in step (e), the reactor was purged using an inert gas to remove excessive ozone. Steps (b)-(e) were repeated 60 times.

After the ozone-based coating process, the uncoated and coated API-2 particles were tested with FT4 rheology meter for flowability (“FF”), cohesive (“cohesive Kpa”), Unconfined Yield Strength (“UYS”) and Major Principle Stress (“MPS”) and Thermogravimetric Analysis (“TGA %”). The testing results are shown in Table 4.

### Fourier Transform Infrared Spectroscopy (FTIR) Analysis

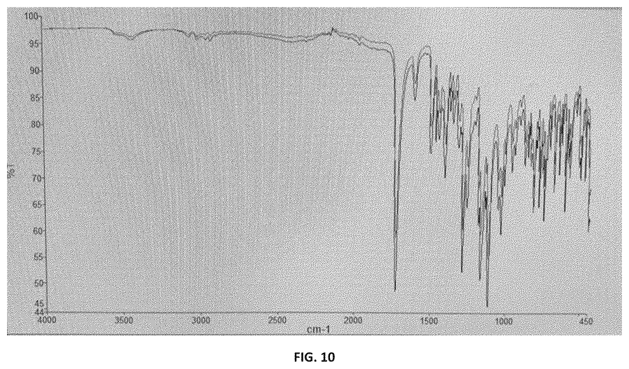

FIG. 10 shows the FTIR spectrum of the coated and uncoated API-2 particles. As shown in FIG. 10, there are no significant change in FTIR signals before and after the TMA-based coating process and ozone-based coating process. This result indicates that there is no change in the chemical bonding information, and that the API was not damaged by the ozone-based coating process.

### Example 5: HPLC Analysis of Ozone Treated Acetaminophen

To test whether ozone treatment will affect the integrity of the API, the API (acetaminophen) was treated with ozone at various concentrations. Here, ozone was created by an ozone generator with various oxygen flow rates. The lower the oxygen flow rate, the high the ozone concentration is. Specifically, the API (acetaminophen) was treated with ozone generated with an oxygen flow rate of 10 sccm, 30 sccm and 50 sccm.

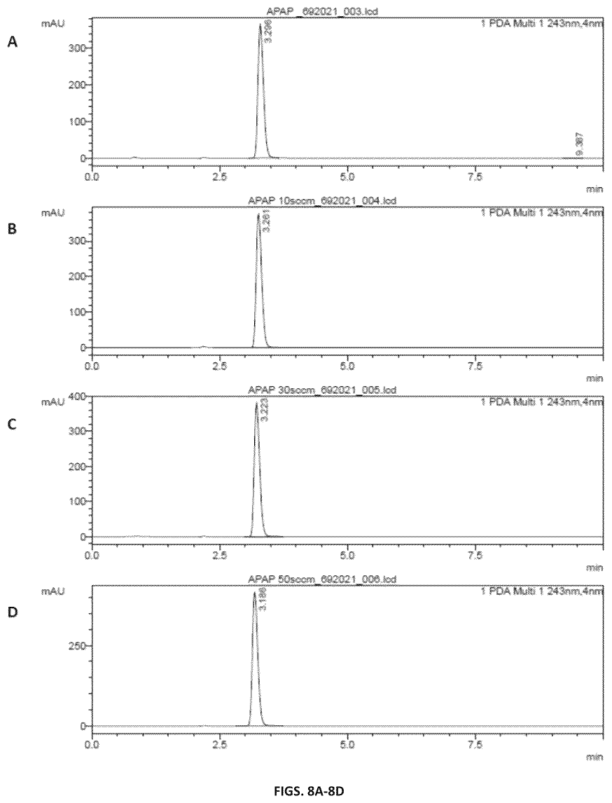

FIGS. 8A-8D show the HPLC analysis of the API (acetaminophen) after ozone treatment. FIG. 8A shows the HPLC analysis of uncoated API (acetaminophen). FIG. 8B shows the HPLC analysis of API treated with ozone generated with an ozone generator with an oxygen flow rate of 10 sccm. FIG. 8C shows the HPLC analysis of API treated with ozone generated with an ozone generator with an oxygen flow rate of 30 sccm. FIG. 8D shows the HPLC analysis of API treated with ozone generated with an ozone generator with an oxygen flow rate of 50 sccm. As shown in FIGS. 8A-8D, there were no new peaks (impurity) generated after ozone exposure with as low as 10 sccm oxygen flow rate (highest O3 concentration)

### Wetting/Dispersity Analysis

The coated API (acetaminophen) particles were dissolved into DI water to test the wetting/dispersity properties of these particles. Uncoated API showed poor wetting/dispersity, API with TMA-based coating showed very good wetting/dispersity, and API with ozone-based coating showed poor wetting/dispersity.

As a control group, API (acetaminophen) was also treated with silane to apply a monolayer of silane. Similar to API with ozone-based coating, the silane-treated API showed poor wetting/dispersity.

### Example 6: Silicon Oxide Coating at 50° C.

API (acetaminophen) was coated with silicon oxide at 50° C. following the methods described in Table 5.

TABLE 5

| | | | | BDIPADS/ | | BDIPADS/ | |
|---|---|---|---|---|---|---|---|
| | | cycles | T | TMA | Water/O₂ | TMA | Water/O₂ |
| BD-9 | APAP | 18 | 50 C. | 0.3 torr | O₂ 100 sccm, | 5 min | 10 min |
| | | | | BDIPADS | 8 torr | | |
| BD-9-BD- | APAP, | 6 | 50 C. | 0.3 torr | H2O | 5 min | 5 min |
| water | BD-9 | | | BDIPADS | 0.5 torr | | |
| BD-9-TMA- | APAP, | 5 | 50 C. | 0.5 torr | O₂ 100 sccm, | 1 min | 5 min |
| O3 | BD-9 | | | TMA | 8 torr | | |

Regarding the ozone-based process for BD-9, in step (a), 3 gram of API (acetaminophen) was loaded to the rotatory reactor (rotating at 10-100 rpm). In step (b), the silicon precursor (BDIPADS) was pulsed into the reactor at about 0.3 torr, with a holding time of 5 minutes and a reaction temperature of 50° C. After the 5-minute holding time, in step (c), the reactor was purged using an inert gas to remove excessive silicon precursors. In step (d), ozone (generated by an ozone generator with an oxygen flow rate of 100 sccm) was pulsed into the reactor to reach 8 torr with a holding time of 10 minutes and a reaction temperature of 50° C. After the 10-minute holding time, in step (e), the reactor was purged using an inert gas to remove excessive ozone. Steps (b)-(e) were repeated 18 times.

Regarding the water-based process for BD-9-BD-water, in step (a), 1 gram of BD-9 with ozone-based coating was loaded to the rotatory reactor (rotating at 10-100 rpm). In step (b), the silicon precursor (BDIPADS) was pulsed into the reactor at about 0.3 torr, with a holding time of 5 minutes and a reaction temperature of 50° C. After the 5-minute holding time, in step (c), the reactor was purged using an inert gas to remove excessive silicon precursors. In step (d), water vapor (0.5 torr) was pulsed into the reactor with a holding time of 5 minutes and a reaction temperature of 50° C. After the 5-minute holding time, in step (e), the reactor was purged using an inert gas to remove excessive ozone. Steps (b)-(e) were repeated 6 times.

Regarding the TMA-ozone based process for BD-9-TMA-O3, in step (a), 1 gram of BD-9 with ozone-based coating was loaded to the rotatory reactor (rotating at 10-100 rpm). In step (b), TMA was pulsed into the reactor at about 0.5 torr, with a holding time of 1 minute and a reaction temperature of 50° C. After the 1-minute holding time, in step (c), the reactor was purged using an inert gas to remove excessive TMA. In step (d), ozone (generated by an ozone generator with an oxygen flow rate of 100 sccm) was pulsed into the reactor to reach 8 torr with a holding time of 5 minutes and a reaction temperature of 50° C. After the 5-minute holding time, in step (e), the reactor was purged using an inert gas to remove excessive ozone. Steps (b)-(e) were repeated 5 times.

### Wetting/Dispersity Analysis

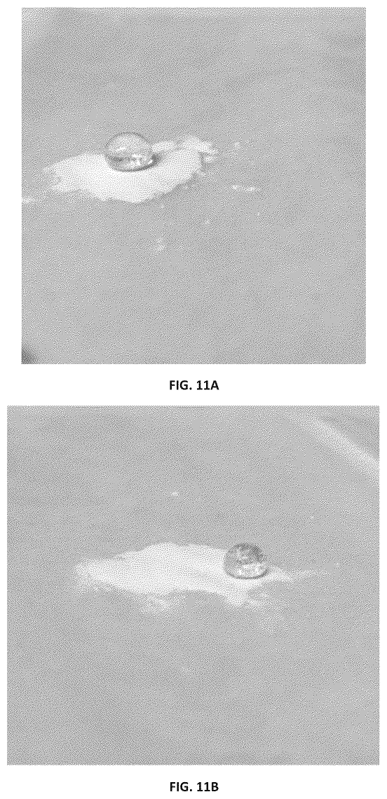

DI water was dropped on to the coated API (acetaminophen) particle pallet to test the wetting properties of these particles. FIGS. 11A-11C show the image of DI water drops on the coated particles. As shown in FIG. 11A, API with ozone-based coating (BD-9) showed poor wetting. As shown in FIG. 11B, API with water-based coating (BD-9-BD-water, no reaction between these two precursors at this temperature) showed poor wetting. As shown in FIG. 11C, API with TMA-ozone based process (BD-9-TMA-O3) showed good wetting. The surface property can be toned by different reaction process.

1. A method of preparing a pharmaceutical composition comprising coated particles comprising an active pharmaceutical ingredient (API) enclosed by one or more silicon oxide layers, the method comprising the sequential steps of:
(a) Providing uncoated particles comprising an API;
(b1) Loading the particles comprising the API into a reactor;
(b2) Applying a vaporous or gaseous silicon precursor to the particles in the reactor by pulsing the vaporous or gaseous silicon precursor into the reactor;
(b3) Performing one or more pump-purge cycles of the reactor using an inert gas;
(b4) Applying an ozone to the particles in the reactor by pulsing the ozone into the reactor;
(b5) Performing one or more pump-purge cycles of the reactor using an inert gas;
(c) Processing the coated particles to prepare a pharmaceutical composition.

2. The method of claim 1, wherein the uncoated particles are crystalline.

3. (canceled)

4. The method of claim 1, wherein the API is an organic compound.

5. (canceled)

6. The method of claim 1, wherein a subset of vapor or gaseous content is pumped out prior to step (b3) and/or step (b5).

7. The method of claim 1, wherein the silicon oxide layer on the coated particles has a thickness in the range of 0.1 nm to 120 nm.

8. The method of claim 7, wherein the silicon oxide layer on the coated particles has a thickness in the range of between 10 nm and 50 nm.

9. The method of claim 1, wherein step (c) comprises combining the coated particles with one or more pharmaceutically acceptable excipients.

10. The method of claim 1, wherein steps (b2)-(b5) takes place at a temperature between 25° C. and 100° C.

11. The method of claim 10, wherein steps (b2)-(b5) takes place at a temperature between 35° C. and 50° C.

12. The method of claim 1, wherein step (b4) comprises a holding time in the range of 1 minute to 1 hour.

13. The method of claim 1, wherein step (b2) comprises a holding time in the range of 1 minute to 1 hour.

14. The method of claim 1, wherein the silicon precursor in step (b2) is Diisopropylamino silane (DIPAS) or 1,2-Bis(diisopropylamino)disilane (BDIPADS).

15. (canceled)

16. The method of claim 1, wherein the ozone in step (b4) is generated using an ozone generator with an oxygen flow rate of 100 sccm.

17. The method of claim 1, wherein step (b1) further comprises agitating the API.

18. A pharmaceutical composition prepared by the method of claim 1.

19. The method of claim 1, wherein the coated particles do not comprise any chloride.

20. (canceled)

21. The method of claim 1, wherein the coated particles exhibit similar or increased hydrophobicity comparing to uncoated particles.

22. The method of claim 1, wherein the silicon oxide coating is free of HCl and Cl.

23. A pharmaceutical composition comprising coated particles comprising an active pharmaceutical ingredient (API) enclosed by one or more silicon oxide layers, wherein the pharmaceutical composition is prepared following the sequential steps of:
(a) Providing uncoated particles comprising an API;
(b1) Loading the particles comprising the API into a reactor;
(b2) Applying a vaporous or gaseous silicon precursor to the particles in the reactor by pulsing the vaporous or gaseous silicon precursor into the reactor;
(b3) Performing one or more pump-purge cycles of the reactor using an inert gas;
(b4) Applying an ozone to the particles in the reactor by pulsing the ozone into the reactor;
(b5) Performing one or more pump-purge cycles of the reactor using an inert gas;
(c) Processing the coated particles to prepare a pharmaceutical composition.

24. The pharmaceutical composition of claim 23, wherein the pharmaceutical composition is free of HCl and Cl.
